(19) United States
(12) Patent Application Publication
(10) Pub. No.: US 2024/0374885 A1
(43) Pub. Date: Nov. 14, 2024

(54) Prism Harmonics

(71) Applicant: Quantum Signature, LLC, Las Vegas, NV (US)

(72) Inventor: Elishia Trainer, Las Vegas, NV (US)

(73) Assignee: Quantum Signature, LLC, Las Vegas, NV (US)

(21) Appl. No.: 18/401,617

(22) Filed: Dec. 31, 2023

Related U.S. Application Data
(60) Provisional application No. 63/436,558, filed on Dec. 31, 2022.

(51) Int. Cl. A61M 37/00

(52) U.S. Cl. CPC A61M 37/0092; A61M 2202/0007; A61M 2210/04

(57) ABSTRACT

This invention marks a significant innovation in the skincare and body care industry, being the first to harness methods for infusing skincare and body care products with a range of sound frequencies. While it prominently features the use of sound and solfeggio frequencies, the methodology is versatile, accommodating a broad spectrum of sound frequencies. Sound frequency infusion is an approach not replicated by any other entity in the market. This process, distinct from conventional frequency infusion methods intended for oral consumption, involves state-of-the-art techniques such as sound emission from playback devices and/or resonation within frequency tuned sound bowls. By being the first in the market to apply such a unique vibrational therapy method in skincare and body care products, ‘Prism Harmonics’ not only elevates daily skincare and body care routines but also introduces novel concepts in vibrational therapy application.

### FIELD OF THE INVENTION

The invention details a specific process for infusing skincare and body care products with sound frequencies, with an emphasis on but not limited by Solfeggio Frequencies. This process involves and precise techniques for integrating sound frequencies into the products, tailored for topical application. The novelty is twofold: a) It provides a novel experience of receiving targeted vibrations, including but not limited to Solfeggio Frequencies, through the direct application of skincare and body care products. This experience is distinct to the user in experiencing sound frequencies from auditory methods or other ambient sound therapies. b) It offers an innovative means of experiencing skincare and body care products as new vehicles for delivering sound frequencies. This concept is unprecedented in the skincare industry, presenting a unique opportunity to users seeking an additional dimension to skin and body care products.

The invention details a non-invasive method for applying vibrational energies to skincare products, emphasizing the technical process of sound frequency integration. This method is detailed in terms of the technical application process, focusing on how vibrational energies are applied and integrated into the products. It introduces a new medium for delivering vibrational exposure in the skincare industry, emphasizing the technical innovation behind this method and its role in diversifying the interaction with vibrational energies in holistic skincare and wellness. This approach not only diversifies the ways in which users can interact with vibrational energies but also underscores the pioneering role of these products in the realm of holistic skincare and wellness.

### BACKGROUND

Nikola Tesla, a pioneer in the study of electromagnetism, once remarked, ‘If you want to find the secrets of the universe, think in terms of energy, frequency, and vibration.’ In this electromagnetic universe, where we visually perceive less than 1% of the frequency spectrum, every biological component vibrates uniquely. James Oschman, PhD, highlights this, stating, ‘In a few decades, scientists have gone from a conviction that there is no such thing as an energy field around the human body, to an absolute certainty that it exists.’ This underlines the role of energy fields in health and disease.

Through the ‘law of sympathetic resonance,’ vibrations matching the environment become amplified and more activated. When biological components are vibrating in a more “natural” and harmonious vibration, the system is able to operate more fluidly and with greater ease. When components of the biological system are vibrating at dissonant, disharmonious vibrations this can be an indicator of greater “stress” on the component. Rupert Sheldrake, a biologist, provides insight into this phenomenon through his morphic resonance theory, suggesting that ‘natural systems inherit a collective memory from all previous things of their kind,’ implying that systems might naturally resonate more positively with harmonious frequencies. In our modern environment, often dominated by artificial frequencies, the idea of reintroducing natural, harmonic frequencies through skincare products aligns with the ‘law of sympathetic resonance’ and the morphic resonance theory, offering a compelling perspective on promoting harmony within biological systems and echoing Albert Einstein's timeless wisdom: ‘Everything is energy. . . Match the frequency of the reality you want and you cannot help but get that reality.

Against the backdrop of a modern environment often dominated by artificial and disharmonious frequencies—from the ubiquitous hum of electronic devices to the relentless drone of machinery—the concept of reintroducing natural, harmonic frequencies through skincare products emerges as an intriguing counterpoint. The hypothesis that engagement with such natural frequencies can offer a reprieve from the omnipresent dissonant tones is supported not only by theoretical frameworks but also by an emerging body of empirical research, underscoring the potential of this innovative approach in skincare. Deepak Chopra, MD, an advocate for alternative medicine, reinforces this idea by stating, ‘Our bodies are not just biochemical machines, but also fields of energy and information.’ This supports the hypothesis that engaging with natural frequencies can offer a reprieve from omnipresent dissonant tones.

Ervin Laszlo, a systems theorist and philosopher of science, adds another layer to this concept: ‘The new concept of the world is an interconnected, coherent cosmos that resonates with the background energy of the universe.’ This suggests that aligning with natural, universal vibrations could be inherently beneficial, underscoring the potential of this innovative approach in skincare. The growing body of empirical research and theoretical frameworks underscores the intriguing possibilities of reintroducing natural, harmonic frequencies. While these frequencies can be explored in numerous contexts, such as enhancing environmental ambience or in innovative product designs, the primary objective is to explore their harmonious integration into our daily lives. This approach is not necessarily about altering biological systems but about exploring how these natural frequencies can be utilized creatively and effectively in various domains.

Coupled with the incredible research regarding the scientific theory that ‘water holds memory’¹²³, there is a unique opportunity to infuse water with vibrational frequencies more aligned with the natural world (such as Solfeggio Frequencies) and to incorporate this water into final skincare formulations, that then carry the vibrational aspects to the user. Additionally, it has been discovered that the skin in fact has “hearing” receptors⁴⁵⁶ and can hear. Supporting this concept, emerging research discusses how plants respond to vibrations⁷⁸⁹¹⁰, suggesting that even mild vibrations with short durations up to one hour can positively affect biological growth, a principle that resonates with our method of infusing skincare products with sound frequencies. For example, sound waves at 1 KHz and 100 dB for 1 hour at a distance of 0.20 meters can significantly promote the division and cell wall fluidity of callus cells in plants¹¹. This also enhances the activity of protective enzymes and endogenous hormones. Another source suggests that sound vibrations can increase yields in several crops and strengthen plant immunity against pathogens¹². These vibrations can also prime plants to make them more tolerant to impending drought. ¹ https://pubmed.ncbi.nlm.nih.gov/17678810/² https://www.mcmill.ca/oss/article/health-and-nutrition/dna-teleporation-really³ https://pubmed.ncbi.nlm.nih.gov/15025874/⁴ https://www.sciencedaily.com/releases/2021/07/210712150320.htm⁵ https://www.scientificamerican.com/article/skin-hearing-airflow-puff-sound-perception/⁶ https://pubmed.ncbi.nlm.nih.gov/32595943/⁷ https://www.theguardian.com/lifeandstyle/2021/jan/11/they-respond-to-vibrations-does-talking-to-plants-actually-help-them-grow⁸ https://www.frontiersin.org/articles/10.3389/fpls.2018.00025/full⁹ https://academic.oup.com/jxb/article/67/15/4483/1749649¹⁰ https://pubmed.ncbi.nlm.nih.gov/27342223/¹¹ https://academic.oup.com/jxb/article/67/15/4483/1749649¹² https://academic.oup.com/jxb/article/67/15/4483/174949?login=false

This approach aligns with the vision of creating products that not only cater to physical skincare needs but also support a holistic wellness paradigm.

The skincare industry often employs marketing terms such as “high vibrational products” without tangible substance behind them. In an industry where terms like “high vibrational products” are often used loosely without any substantiation, the present innovation represents a departure marked by substance. This is the first time skincare and body care products have been infused with targeted sound frequencies using our simple yet novel methods in order to provide substance to the phrase, “high vibrational products”.

The integration of quantum physics principles and the profound philosophies from the Upanishads¹³ (sacred Sanskrit texts dating back thousands of years) further offers a compelling backdrop for this invention. It reflects the complex interaction between energy, frequency, and consciousness, resonating with the application's use of sound frequencies in skincare. This convergence of modern scientific understanding with ancient wisdom supports the invention's conceptual basis, where the influence of vibrational energy on user experience is central. This alignment enhances the depth and novelty of the skincare products, positioning them at the intersection of science, wellness, and holistic understanding. ¹³ https://science.thewire.in/society/history/erwin-schrodinger-quantum-mechanics-philosophy-of-physics-upanishads/

### Solfeggio Frequencies

Solfeggio Frequencies are believed to resonate deeply with biological systems. The innovative use of these frequencies in skincare and body care products is supported by various scientific studies. Research indicates that specific sound frequencies, like 528 Hz¹⁴, can influence biological processes, such as testosterone production in the brain, or positively impact cognitive and endocrine responses in organisms like zebrafish¹⁵. The principle of ‘Sympathetic Resonance’ underlies these effects, where matching vibrational frequencies can amplify inherent vibrations in water, living tissues, and material objects¹⁶. ¹⁴ https://pubmed.ncbi.nlm.nih.gov/30414050/¹⁵ https://pubmed.ncbi.nlm.nih.gov/37119977/¹⁶ https://sciencedemonstrations.fas.harvard.edn/presentations/shattering-wineglass

Within this framework, the proposed invention serves as a medium to infuse everyday skincare and body care products with solfeggio and/or other sound frequencies, providing users with the opportunity to enhance their environment with tones that have been associated with a resonate connection with nature. By embedding these frequencies into body care items, the invention offers a practical and accessible means to integrate the benefits of these natural frequencies into one's daily routine. Thus, the innovation stands as a testimony to the potential of combining ancient wisdom with contemporary technology to promote well-being in the midst of a cacophonous world.

Dr. Glen Rein's study highlights the positive effects of different music types, including Gregorian chants related to Solfeggio Frequencies, on human DNA. This research suggests that sound frequencies, like Solfeggio frequencies, have significant health impacts. Other studies indicate profound mental, emotional, and physical effects of Solfeggio frequencies, aligning with therapeutic applications in skincare. This evidence underscores the relevance of Solfeggio frequencies in health and wellness products¹⁷¹⁸¹⁹. ¹⁷ https://www.researchgate.net/publication/333852911_Sound_Healing_using_Solfeggio_Frequencies¹⁸ https://www.forbes.com/sites/forbestechcouncil/2022/12/21/think-youve-heard-it-all-new-research-into-the-science-of-sound-proves-otherwise/?sh=4f51fb1c684b¹⁹ https://www.bettersleep.com/blog/science-behind-solfeggio-frequencies/

### Sound Therapy

The application also draws attention to the broader field of sound therapy, acknowledging its historical use with ancient Himalayan bowls, Earth gongs, tuning forks, and other vibration instruments, all which find their power in sound frequencies and/or Solfeggio Frequencies. Sound therapy, also leveraging vibrations such as ‘Om’ or other vocal chants, has been reported to reduce stress, emotional, and physical pain, contributing to overall well-being²⁰. ²⁰ https://www.apa.org/monitor/2013/11/music

The scientific underpinnings of sound frequency infusions find support in the work of researchers like Dr. Lee Bartel²¹ and Joanna McEwen²²²³. Dr. Lee Bartel's research in vibroacoustic therapy underscores the therapeutic applications of sound vibrations. Joanna McEwen's exploration of sound therapy and its harmonizing effects on the body parallels the holistic approach of this invention, suggesting the broader applications of sound vibrations in health in general. ²¹ https://vibroacoustic.net/music-medicine-sound-at-a-cellular-level-dr-lee-bartel-tedxcollingwood/²² https://https://youtu.be/SLSsqCl7NIE?si=M3m3qJG4SWWHWGbE²³ https://www.healingfrequenciesmusic.com/what-are-sound-essences/

### Cymatics

Cymatics²⁴, the study of visualizing audio frequencies, offers fascinating insights into the scientific intersection of sound, matter, and form. When various materials like liquids, powders, or particles are exposed to sound frequencies, they organize into complex, often geometric patterns, illustrating the physical manifestation of vibration. This phenomenon, first observed and named by Hans Jenny in the 1960s, is based on the principle that sound waves, as a form of energy, exert a force on the medium through which they pass. These patterns vary depending on the frequency and amplitude of the sound, as well as the properties of the medium. Cymatics provides a compelling visual representation of how sound frequencies can influence and organize matter²⁵, underscoring the profound impact that sound can have on the physical world. This field has implications in various areas, including acoustics, sound therapy, and even material science, as it offers a unique perspective on the dynamic interaction between sound and physical forms. ²⁴ https://jonmeyofcuriosity.net/pages/what-is-cymatics-how-to-explained²⁵ https://cymascope.com/micro-cymatics/

### Water's Role

Water is undeniably fundamental to life on Earth, serving as the very essence of our existence. From the oceans where life originated to the amniotic fluid enveloping humans in their earliest stages, water's significance cannot be overstated. Comprising 70% of the human body, it is the lifeblood that sustains us. Water is a fundamental element of our existence, comprising a significant portion of the human body. It has the unique ability to retain vibrations, including those generated by sound. This remarkable capacity aligns with our innovation's focus on infusing water with vibrational frequencies, such as Solfeggio Frequencies, and incorporating it into skincare formulations. In essence, water can retain a memory of the molecular properties it encounters, echoing the sentiment expressed by physicist Melvin Vopson that ‘Information is Energy.’

Esteemed scientists, including Dr. Viktor Schauberger²⁶, Dr. Masaru Emoto²⁷, and Yolène Thomas²⁸, have studied the influence of vibrations on water. Their research emphasizes water's ability to capture and transmit vibrational information, a central principle in our product's design. These findings validate our approach of infusing skincare products with specific frequencies, leveraging water as a medium to deliver therapeutic benefits. Thomas's study delves into water's remarkable ability to retain and transmit vibrational information, a core principle underpinning our product. Her findings affirm our approach of infusing water in skincare products with specific frequencies, reinforcing the notion that these frequencies can be preserved and conveyed by water for therapeutic benefits. This aligns seamlessly with our innovative concept, where frequency-infused water in our products plays a pivotal role in delivering wellness advantages to users. ²⁶ https://borderlandsciences.org/journal/vol/35/n03/Zock_on_Victor_Schauberger.html²⁷ https://pubmed.ncbi.nlm.nih.gov/15025874/²⁸ https://pubmed.ncbi.nlm.nih.gov/17678810/

A parallel can be drawn between our invention's concept of water retaining vibrational frequencies and Dr. Luc Montagnier's groundbreaking research²⁹, as detailed in McGill University's Office for Science and Society. Montagnier's investigation explores the intriguing possibility of water retaining electromagnetic signals from substances like DNA, even after substantial dilution. This concept, while groundbreaking, complements our method of infusing skincare products with specific frequencies, suggesting water's potential to uphold and transmit these frequencies and to hold these new signatures even at substantial dilution. Montagnier's hypothesis concerning water's information-carrying capacity lends credence to the innovative approach of our frequency-infused topical skincare and body care products. ²⁹ https:www.mcgill/ca/oss/article/health-and-nutrition/dna-teleportation/really

### The Use of Spiral Vortex Movement

Part of our innovative process includes the option of incorporating the principle of movement to enhance the efficacy of our product component(s) frequency infusion. Drawing upon the dynamics of fluid motion, we apply spiral vortex movement to the infusion process, aligning with the natural rhythms and flows found in nature³⁰³¹. This movement is not merely physical agitation; it is a deliberate, calculated action that optimizes the interaction between the product ingredients and the infused sound frequencies. The specific movement patterns employed are based on principles such as vortex dynamics, which have been observed to significantly alter the properties of liquids. This approach to incorporating movement into our infusion process ensures a deeper, more harmonious integration of sound frequencies with the product's base materials, leading to a product that is not only effective in its skincare properties but also in alignment with natural processes. ³⁰ https://sdmiramax.edu/sites/default/files/2023-05/Structured%20Water.pdf³¹ https://www.scirp.org/journal/paperinformation♭paperid=75862

The use of specific vortex movement from either the magnetic stirrer or the sound bowl swirling in our method represents a groundbreaking step in the field of skincare, leveraging physical dynamics to unlock new potentials in product formulation. Recent advancements in the understanding of water's physical properties, notably by Dr. Gerald Pollack³² of the University of Washington, provide a compelling scientific framework for our patent's methodology. Dr. Pollack's research of water reveals the profound influence of mechanical perturbations, such as vortexing, on water's structure. This research supports our invention's use of vortex movement to enhance the structuring of water in skincare and body care products. The process of vortexing, as elucidated by Dr. Pollack, is recognized as a powerful method to increase the organization and alignment of water molecules, thereby potentially enhancing the water's functional properties in our formulations. This novel approach to water structuring, informed by cutting-edge scientific insights, underpins the unique efficacy of our products in delivering skincare benefits. ³² https://www.hexagonalwater.com/jerald_pollack.html

### Specialized Skin Receptors

Specialized Skin Receptors: The human skin is equipped with specialized sensory receptors³³³⁴ responsive to vibrations, offering an additional avenue for sensory exposure beyond traditional auditory channels. A study featured in Scientific American highlights the skin's ability to contribute to auditory perception³⁵. Recent research further underscores the skin's ability to perceive auditory stimuli, highlighting a dynamic interaction with vibrational skincare products. Studies emphasize the role of skin mechanoreceptors like Meissner and Pacinian corpuscles, which detect and relay vibrational signals to the brain, influencing cognitive and sensory functions. ³³ https://www.sciencedaily.com/releases/2021/07/210712150320.htm³⁴ https://www.sciencedaily.com/releases/2009/12/091208083524.htm³⁵ https://www.scientificamerican.com/article/skin-hearing-airflow-puff-sound-perception/

Advanced Sensory Research: Whole Body Vibration (WBV)³⁶ research demonstrates the sensory impact of vibration on the brain via skin mechanoreceptors. Additionally, groundbreaking research by Pohang University of Science & Technology (POSTECH)³⁷ has developed a skin-attachable acoustic sensor that surpasses human ears' auditory sensitivity. This sensor reveals the skin's remarkable ability to directly interact with sound and vibration, expanding our understanding of how the skin perceives and responds to external auditory stimuli. ³⁶ https://pubmed.ncbi.nlm.nih.gov/32595943/³⁷ https://www.sciencedaily.com/releases/2022/07/220701102740.htm

Innovative Application: Integrating these insights into our product's design, we hypothesize that the skin's interaction with our specially crafted skincare products, infused with structured water created through specific vibrations, could be more dynamic and responsive. The incorporation of Solfeggio Frequencies in our skincare emulsions is intended to harmonize with the skin's recently recognized sensory capabilities. This has the potential to enhance the skin's interaction with and benefits from the vibrational qualities of our products, offering a unique and enhanced user experience in skincare.

### PATENT CITATIONS

US Patent Application #20180092449
   a. Publication Date: Apr. 5, 2018
   b. Title: “Systems, Devices, And Methods For Generating Auditory Alerts/Indications Using Resonant Sonic Motor”
   c. Inventor(s): Scott Straka et al.
   d. Assignee: L'Oréal
   e. Summary: This application describes a personal care appliance designed to generate auditory alerts or indications using a sonic motor operating at a resonant frequency. The device includes a motor assembly for oscillating a brush head, with various features such as a brush head replacement timer, a battery power indicator, and a dispenser for a topical agent. It also allows for communication with a central device like a mobile phone to customize the auditory tone emitted upon certain events.
U.S. Pat. No. 11,065,416
   a. Publication Date: Jul. 20, 2021
   b. Title: “Therapeutic Device”
   c. Summary: This patent is for a therapeutic device that integrates aroma, sound, vibration, and color therapy. It includes components like an enclosure, oil-diffusing and frequency-infusing devices, a frequency-tone generator, ambient lights, vents, and a controller. The device aims to combine various therapeutic methods into one appliance for enhanced user experience.
US Patent Application #20210076871
   a. Publication Date: Mar. 18, 2021
   b. Title: “Infusing Apparatus and Process and Method for Using the Same”
   c. Summary: This application describes a process and device for brewing coffee using pressure differential techniques. It emphasizes the use of pressure differentials in the infusion process, primarily focusing on brewing coffee.
US Patent Application #20230397752
   a. Publication Date: Dec. 14, 2023
   b. Title: “Beverage Infusing Systems”
   c. Summary: This patent application details a system for infusing beverages using ultrasonic waves. It highlights the advantage of using ultrasonic energy to increase the efficiency of the infusion process.
U.S. Pat. No. 10,941,061
   a. Publication Date: Mar. 9, 2021
   b. Title: “Therapeutic Frequency Imprinting Device”
   c. Summary: This patent describes a device for imprinting therapeutic frequencies into a carrier material or substance, such as water. It is pertinent for infusing frequencies for therapeutic purposes.
US Patent Application #20070157791
   a. Publication Date: Jul. 12, 2007
   b. Title: “Methods for Infusing Matter with Vibration”
   c. Summary: This application discusses methods for infusing water with vibrations. It covers techniques for measuring the vibration infused in water and the potential effects on consumers.

### DETAILED DESCRIPTION OF THE INVENTION

This sound frequency infusion process is accomplished through various methods, including but not limited to exposing the product component(s) in proximity to the emitted sound from a playback device or a frequency tuned sound bowl instrument, or a human voice, allowing the frequency to be absorbed by the product component(s) to at least partially used in a skincare and/or body care final product formulation. In the latter method, the product component(s) is contained within the sound bowl, which is struck to resonate the frequency into the product component(s). The skin's specialized sensory receptors, responsive to vibrations, offer a novel exposure pathway for exposure to frequency and vibration through skincare and body care topical products.

Our invention distinctly diverges from existing frequency infusion methods in water, which are primarily designed for oral consumption or for therapeutic use. Our methods seek to provide an alternative method for the user to experience vibrational exposure beyond consumption, hearing, or ambient use. Unlike other methods involving frequency infusion of water, our invention is specifically tailored for topical application of complex liquid emulsions containing but not limited to: water, oils, plant botanicals, peptides, and other active skincare or body care ingredients. The core intention of our unique process is not for the user to ingest the frequency infused water, but rather to apply the frequency infused product directly to their skin. This method leverages the skin's ability to be exposed to these frequencies, offering a novel pathway for the benefits of vibrational exposure. Our approach utilizes the principle of vibrational resonance not through ingestion but through direct skin contact, providing a distinctive modality for experiencing the benefits of infused frequencies. This significant difference in application and interaction with the human body sets our invention apart in the field of vibrational frequency infusion and liquid mediums. No other skincare or body care brand is infusing their products with sound frequencies. This is new for the skincare and body care industry as well.

In our innovative approach to frequency infused skincare products, we recognize the potential of not only water-based but also oil-based products to interact with vibrational frequencies. This inclusion of liquid oil mediums aligns with contemporary scientific understanding³⁸, underscoring the versatility and potential efficacy of our invention. By leveraging the inherent properties of both water and oils, our frequency-infusion process promises a comprehensive and multifaceted approach to enhancing skincare & body care products, delivering the benefits of vibrational exposure across a wider range of product formulations. ³⁸ https://static.youngliving.com/en-US/PDFS/Bioenergetics_Handout.pdf

Our innovative approach in infusing skincare products with sound frequencies goes beyond mere physical enhancement. We firmly believe that this unique exposure to frequencies through the skin opens a gateway for the energy body to access and with a deeper level of information stored within these energies. This offers a connection between the body's physical and energetic realms.

The present disclosure is directed generally to liquids infused with operative frequencies. The frequencies may vary depending on application. In certain embodiments, the frequencies may be selected from 396 hz, 417 hz, 528 hz, 639 hz, 741 hz, 852 hz and 936 hz and combinations thereof. The vibrations may also include positive vocal affirmations at these frequencies as well. The liquids to be infused may include water and water-based liquids, as well as any and all plant oils, fixed oils, used for skincare or body care but not limited to these uses. Some examples, but not limited to, include grapeseed oil, almond oil, prickly pear oil, baobab oil, kiwi oil, rosehip oil and shea butter. In certain embodiments, the liquids may comprise a mixture of two or more of the above-listed liquids and/or paired with other unmentioned ingredients.

### Time Duration of Exposure, Volume

The variable time duration for sound frequency exposure within this patent application is intentionally left open to accommodate a diverse range of practices and applications. Scientific research, exemplified by studies such as those conducted at Harvard³⁹ where a wine glass was shattered after frequency exposure. Others have repeated this experiment and shattered the glass after a mere 30 seconds of exposure to a 120-decibel volume, demonstrates the significance of resonant frequency exposure at shorter intervals. Conversely, the field of sound therapy often recommends exposure durations for humans spanning from 3 to 15 minutes to harness therapeutic benefits⁴⁰⁴¹. In historical contexts, Montaigne engaged in the infusion of water for 18 hours⁴², while Emoto's practices extended to months. Science is also showing that plants can respond to mild vibrations almost immediately. While embracing this wide spectrum, practical empirical evidence derived from our own experiments suggests that, for many applications, a duration range of 3-15 minutes is often sufficient. This period aligns with a volume audible to the human ear without causing discomfort. The deliberately open-ended approach herein ensures flexibility and adaptability in the application of sound frequencies to product components, contributing to the versatility of this innovative method and allowing for flexibility as science evolves and emerges. ³⁹ https://sciencedemonstrations.fas.harvard.edn/presentations/shattering-wineglass⁴⁰ https://www.chiangmaiholistic.com/7.83hz.php⁴¹ https://www.today.com/health/what-are-sound-baths-experts-discuss-practice-health-benefits-t234137⁴² https://www.i-sis.org.uk/DNA_sequence_reconstituted_from_Water_Memory.php

### Integration of Entrainment Principle in Sound Frequency Infused Skincare

The concept of entrainment⁴³, a phenomenon observed in physics and biology, plays a crucial role in the application of sound frequency-infused skincare and body care products. Entrainment, as initially observed by Christiaan Huygens in the 17th century, refers to the synchronization of rhythmic cycles due to the interaction of frequencies⁴⁴. ⁴³ https://www.ncbi.nlm.nih.gov/pmc/articles/PMC9010850/⁴⁴ https://www.open.ac.nk/Arts/experience/InTimeWithTheMusic.pdf

### Description of the Infusion Process

Innovative Integration: The core innovation lies in integrating specific sound frequencies, such as Solfeggio frequencies, into skincare products. This method is based on the theoretical concept that sound frequencies can interact with various mediums, potentially leading to unique product characteristics.

Process Mechanism: The process involves exposing skincare formulations to selected sound frequencies during production. The principle of entrainment is applied as a theoretical framework to explain the potential interaction between sound frequencies and the product ingredients.

Potential for Synchronization: While the application does not claim specific health benefits, it is based on the hypothesis that the entrainment process might influence the properties of the skincare product. This is grounded in the theoretical understanding of how external frequencies can interact with different mediums. The principle of entrainment suggests that the sound frequencies could have therapeutic effects, such stress reduction at the cellular level or enhanced repair processes. This aligns with the concept that certain frequencies, like those in the Solfeggio range, possess inherent healing properties⁴⁵. ⁴⁵ https://www.researchgate.net/publication/327439522_Effect_of_528_Hz_Music_on_the_Endocrine_System_and_Autonomic_Nervous_System

Focus on Methodology: The emphasis of this patent is on the methodological innovation of incorporating sound frequencies into skincare products. The application details the process of sound frequency infusion, showcasing its novelty in the skincare industry.

Avoiding Health Claims: This application strictly adheres to patent guidelines by focusing on the process and its theoretical underpinnings without making direct claims about health or therapeutic benefits.

### Versatility of Infusion Process

Our method of infusing skincare and body care products with sound frequencies is designed for versatility. It can be effectively applied at different stages of the product's lifecycle, either by infusing water with sound frequencies prior to its incorporation into the product formulation or by applying sound frequencies to the finished product. This flexibility allows for the adaptation of the infusion process according to the specific product characteristics and manufacturing requirements, ensuring the effectiveness of the frequency infusion irrespective of the stage at which it is applied. The infusion process is applicable to both the full emulsions and their individual components.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refers to at least one of something selected from the group consisting of A, B, C . . . and N, the text should be interpreted as requiring only one element from the group, not A plus N, or B plus N, etc.

### HiViBe33's Chakra Tuned® Products

Many products under HiViBe33's registered trademark ‘Chakra Tuned’ is paired with a specific Solfeggio Frequency. While not necessary for the effectiveness of the invention or product, this pairing aligns each frequency with a chakra and a corresponding chakra intention, fostering a deeper connection between the product and its conceptual use. The intention is to create products that resonate with specific aspects of wellness as traditionally associated with each chakra, thus offering a unique vibrational experience:
Root Chakra|396 hz Solfeggio Frequency
Sacral Chakra|417 hz Solfeggio Frequency
Solar Chakra|528 hz Solfeggio Frequency
Heart Chakra|639 hz Solfeggio Frequency
Throat Chakra|741 hz Solfeggio Frequency
Third Eye Chakra|852 hz Solfeggio Frequency
Crown Chakra|936 hz Solfeggio Frequency

Our approach is rooted in the concept of sympathetic resonance, where exposure to these specific vibrations is designed to enhance the experiential quality of the products. By incorporating these frequencies, we aim to imbue our skincare products with a harmonious vibrational character that aligns with the traditional understanding of chakras and their associated frequencies. We however are not limited by only these seven Solfeggio Frequencies.

### Summary of Patentability

In this section, we aim to demonstrate how our invention, “Sound Frequency Infused Skincare and Body Care Products,” meets the essential standards for patentability.

Novelty: This invention introduces a unique method in skincare and body care, utilizing sound frequencies for an innovative infusion process. Unlike existing market offerings, this method focuses on integrating sound vibrations into skincare products, offering a distinctive user experience. It differs from traditional practices in skincare and wellness by providing an alternative way for users to interact with sound vibrations through topical application. The process is designed to enrich the user's skincare routine with the inclusion of sound frequencies, differentiating it from conventional tactile vibrational therapies.

Non-obviousness: The integration of Solfeggio Frequencies into skincare and body care products represents a non-obvious innovation in the field. This unique approach diverges significantly from the conventional methods prevalent in skincare, which predominantly focus on physical and chemical properties. Our invention transcends standard practices by incorporating a novel aspect of sound frequency infusion, a concept unexplored in current skincare technologies. This inventive step, which merges elements of sound therapy and skincare, is not an obvious progression for experts in the field and therefore meets the criteria for non-obviousness in patentability. It refrains from making specific biological claims about the skin's interaction with these frequencies, focusing instead on the unique method of their application and potential experiential benefits. Second, it is non-obvious to be able to experience targeted vibrations and frequencies through skincare and body care.

Utility: The proposed invention, ‘Sound Frequency Infused Skincare and Body Care Products,’ offers multifaceted practical utility, distinguished by the following attributes:
i. Innovative Infusion Method: At the core of its utility is the innovative technique of integrating sound frequencies, particularly Solfeggio Frequencies, into skincare and body care products, both with and without the use of spiral vortex. This method is designed to enhance the product experience by offering a unique interaction with sound vibrations through topical application. This approach not only diversifies the user's engagement with skincare products but also introduces a new dimension to the application of sound therapy in topical skin and body care.
ii. Enhanced User Experience: Specifically tailored for topical application, this invention deviates from conventional skincare paradigms. It leverages the skin's sensory capabilities to interact with vibrational frequencies, potentially offering an enriched and distinctive skincare experience. This aspect emphasizes the holistic approach to skincare, where the focus extends beyond traditional tactile and olfactory experiences to include the novel dimension of vibrational interaction.
iii. Versatile Formulation Compatibility: Demonstrating formulation flexibility, the process is adeptly adaptable to a diverse range of skincare products, including both water and oil-based formulations. This versatility ensures that the benefits of sound frequency infusion can be experienced across a wide spectrum of skincare needs and preferences, catering to a broad consumer base.

Enablement: The patent application provides a comprehensive description of the frequency infusion process. This includes the detailed methodology of using Sound Frequencies, the equipment employed, and the rationale behind choosing specific frequencies. Such detailed disclosure ensures that a person skilled in the art would be able to replicate our innovative process without undue experimentation.

Subject Matter Eligibility: Our invention aligns with the criteria for patentable subject matter, presenting a tangible method and product. It transcends abstract concepts by offering a practical application of sound frequency in skincare, a concrete process distinct from theoretical ideas. This approach, centered on a physical product and specific method of frequency infusion in a topical application, adheres to patent eligibility guidelines, ensuring that it is rooted in a real-world application and not merely an abstract or natural phenomenon.

In conclusion, the invention “Sound Frequency Infused Skincare and Body Care Products” satisfies the criteria for patentability. It is a novel, non-obvious, and useful invention, adequately described for replication by skilled individuals, and falls within the acceptable scope of patentable subject matter.

In the context of this patent application, the term ‘product component(s)’ shall refer to ingredients or substances used in the creation of final skincare and body care products. This includes, but is not limited to: water, oils, esters, hydrosols, alcohol, plant botanicals, full or partial emulsions, to be used in the final product and/or including the final product itself. These components are the primary subjects of the infusion process described herein, where they are exposed to various sound frequencies. The term ‘product component’ is intended to be inclusive of a wide range of materials commonly used in skincare and body care formulations, allowing for flexibility and adaptability in the application of the described methods.

### PROCESSES

### Process 1: Playback Device Infusion

The primary objective of the speaker process is to expose ‘product component’ to specific sound frequencies, played from a playback device for a duration of at least 3 minutes while the beaker holding the product component is either placed on a solid surface or alternatively can be placed on a magnetic stirrer for further enhancement, where the magnet is placed inside the liquid and a spiral vortex is created simultaneously throughout the frequency exposure process. The spiral vortex is not necessary for the infusion but is an enhancement.

In the infusion process, the volume of the sound frequencies should be audible to the human ear, without necessitating a specific decibel level.

### Setup

Illustrated by FIG. 1A, the speaker infusion process involves placing a speaker on top of a glass beaker containing the skincare emulsion. The speaker faces towards the ‘product component’ from the liquid. Sterile instruments are used, and the work area is maintained in a sterile condition. A glass beaker is not a necessary component.

### Playback Device Details

Any playback device that emits sound can be used for this process.

### Frequency Selection

Sound Frequencies are played through any numerous playback devices. MP3 files, Records, and/or musical or frequency or meditation apps (not limited to only these choices) that are equipped to play the chosen sound frequencies. The sound plays throughout the infusion process. Here, one may choose any sound frequency including a Solfeggio Frequency, a recorded vocalization, or and/a recorded or natural sounds from the natural environment.

### Infusion Process

The skincare and body care product components are exposed to the selected sound frequencies from the speaker turned on and playing the frequencies with or without the magnetic stirrer creating a visible spiral vortex for a recommended duration of at least 3 minutes. This timeframe is informed by current expert opinions and preliminary data. However, recognizing the evolving nature of research in this field, such as insights from Dr. Luc Montagnier's work⁴⁶, our application is designed to accommodate longer durations of this process, up to 18 hours, should future research support such a need. This approach ensures our method remains adaptable to new scientific discoveries and best practices in vibrational therapy. The sound frequency is audible to the human ear. ⁴⁶ https://www.i-sis.org.uk/DNA_sequence_reconstituted_from_Water_Memory.php

### Diagram of Process 1: Playback Device Infusion (FIG. 1A)

### Process 2: Sound Bowl Infusion

### Objectives

The primary objective of the sound bowl infusion method is to infuse product component(s) with specific sound frequencies and vibrations, from sound bowl instruments in play to the product component(s). The liquid may also be placed in a protective container, such as a petri dish or similar vessel, to prevent direct contact with the sound bowl. However, this is an optional enhancement and not a strict requirement of the process. The product component(s) can also be in close proximity to the sound bowl while the sound bowl is played.

The exposure period to the frequencies is set at least 3 minutes, during which the sound bowl is consistently played for the duration of the infusion. The product component will have a visible swirling motion as the instrument is played, emulating a spiral like vortex, and the sound frequency is audible to the human ear. If the product is placed in proximity to the sound bowl rather than inside the sound bowl, the spiral vortex is not needed.

### Setup

Illustrated by FIG. 1B, illustrates the process where the product component(s) is inside the sound bowl instrument. A proper mallet is necessary to strike the sound bowl instrument in order to play it.

### Infusion Process

The sound bowl is activated by striking it with an appropriate mallet, initiating the infusion of the liquid with the sound frequencies' vibrations. It is played to maintain resonance for a recommended duration of at least 3 minutes. This timeframe is informed by current expert opinions on sound therapy and preliminary data. However, recognizing the evolving nature of research in this field, such as insights from Dr. Luc Montagnier's work⁴⁷, our application is designed to accommodate longer durations, up to 18 hours, should future research support such a need. This approach ensures our method remains adaptable to new scientific discoveries and best practices in vibrational therapy. ⁴⁷ https://www.i-sis.org.uk/DNA_sequence_reconstituted_from_Water_Memory.php

### Frequency Selection

The appropriately frequency tuned sound bowl instrument is chosen based on the specific frequency desired for the infusion. Sound bowl instruments come in different shapes, sizes, materials, and frequency tunings. All types are acceptable. If one wants to infuse the product or components of the product or water with a Solfeggio Frequency, they may select a sound bowl instrument tuned to that frequency. That is all that is needed. There are numerous sound bowl instruments tuned to different frequencies, usually designated by the sound bowl musician or the manufacturer. The sound bowl will be played for the duration of the infusion process. The frequency infusion remains constant for each individual product, providing a consistent and controlled infusion process.

### Diagram Process 2: Sound Bowl Infusion (FIG. 2B)

### Process 3: Human Voice Vocalizations

### Objectives

The objective of this method is to infuse product components with specific sound frequencies using human voice. Unlike the sound bowl method, this involves direct vocalization onto the product.

### Setup

The setup involves a controlled environment where vocal sounds are produced directly towards the product components.

### Infusion Process

The process involves exposing the product components to live human vocal sounds, such as singing or speaking, for a set duration. This direct exposure embeds the acoustic properties of the voice into the products.

### Duration

The recommended exposure duration is at least 3 minutes, with flexibility for adjustment based on evolving research and findings in the field.

### Conclusion

Post infusion, the product components, now enriched with the vibrational qualities of the human voice, are ready for integration into skincare products.

1. Method for Infusing Skin and Body Care Products with One of the Seven Solfeggio Frequencies.
A method for infusing skincare and body care products with one of the seven designated Solfeggio Frequencies, such as: 396 hz, 417 hz, 528 hz, 639 hz, 741 hz, 852 hz, and/or 936 hz. Comprised of selecting a product component(s) and applying one of the seven Solfeggio Frequencies to the product component using a frequency process outlined above; with or without the use of a spiral vortex to enhance the vibrational infusion.

2. Method for Integrating Sound Frequencies into Skincare Products.
The process involves selecting a product component(s) and applying sound frequencies to the product component using one of the frequency processes outlined above; with or without the use of a spiral vortex. The method includes maintaining the frequency application (and potential spiral vortex movement) for the recommended duration of at least 3-15 minutes, with the understanding that this duration may be adjusted in future applications as scientific understanding evolves.
This method encompasses the use of sound frequencies ranging from 1 Hz to 20,000 Hz, ensuring complete coverage of frequencies for skincare and body care product frequency infusion.
The claim includes utilization of these frequencies in any combination or sequence, providing a versatile and all-encompassing approach to enhancing product properties through sound frequency infusion.

3. Topical Skincare Products with Infused Sound Frequencies.
A range of skincare or body care products, developed for topical application, that incorporate sound frequencies. The claim specifies the method by which these products provide a novel way of experiencing frequencies through topical application, distinct from auditory or ambient exposure. This claim specifies that the products offer a unique way of experiencing frequencies through topical application, made distinct by the effect of sound frequency infusion.

4. Method for Harnessing Water's Vibrational Resonance in Skincare Product Formulation.
A method utilizing both the vibrational storage capacity of water and the enhancement provided by spiral vortex movement in skincare and body care products. The process includes infusing water with sound frequencies and spiral vortex movement, with the sound audible to the human ear, then integrating this vibrationally enhanced water into skincare and body care formulations. This approach imparts unique properties to the final product, leveraging both the vibrational resonance and the dynamic effects of spiral vortex movement.

5. Method for Infusing Skin and Body Care Products with Sound Frequencies Using Sound Bowls and Other Instruments.
A method for infusing skincare and body care products with frequencies generated by any of the following but not limited by: sound bowls, gongs, tuning forks, pianos, violins, cellos, being played to resonate sound. This method is characterized by utilizing sound instrument vibrations, either through direct or proximity-based infusion, to impart unique vibrational qualities to skincare products. If the sound produced by an instrument is audible to the human ear, it is deemed sufficient for the product components to be within the effective range for infusion. This approach ensures that the sound vibrations, emanating from instruments like gongs or tuning forks, interact with the product ingredients even without direct contact, leveraging the principle that audible frequencies are capable of imparting beneficial vibrational properties to the products. Exposing skincare and body care product ingredients to these sounds for an initial recommended duration of at least 3 minutes, with the understanding that this duration may be adjusted in future applications as scientific understanding evolves.

6. Method for Infusing Skin and Body Care Products with Sound Frequencies Using Direct Vocal Frequencies.
A method for infusing skincare and body care products with frequencies from live human vocalization, with or without the use of a spiral vortex, comprising:
1. Directly exposing product ingredients to live human vocal sounds;
2. Conducting the infusion in a setting where the vocal sounds are produced in real-time and transmitted to the product ingredients;
3. Utilizing the unique vibrational qualities of the human voice;
4. If the sound produced by an instrument is audible to the human ear, it is deemed sufficient for the product components to be within the effective range for infusion.
5. Recommending an initial exposure duration of at least 3 minutes, adjustable based on scientific developments.

7. Method for Infusing Skin and Body Care Products with Frequencies Using Sound Played from a Playback Device.
A method for infusing skincare and body care products with frequencies emitted from a playback device, with or without the use of a spiral vortex, comprising:
a. Positioning a playback device in proximity to skincare and body care product ingredients contained in a suitable vessel;
b. Emitting sound frequencies from the playback device for a specific duration to infuse the product ingredients with these sound frequencies.
c. Exposing skincare and body care product component(s) to these sounds through a playback device for an initial recommended duration of at least 3-15 minutes, with the understanding that this duration may be adjusted in future applications as scientific understanding evolves.

8. Method for Infusing Skin and Body Care Products with Sound Frequencies Through Natural and/or Human-Modified Environmental Sounds.
A method for infusing skincare and body care products with natural environmental sounds, which may include original or human-modified sounds such as whale songs, desert sounds, or other environmental acoustic phenomena, with or without the use of spiral vortex. Utilizing recordings of environmental sounds, which can be either in their natural state or modified through human intervention for desired acoustic properties. Exposing skincare and body care product ingredients to these environmental sounds through a playback device, ensuring effective transmission of their unique frequency characteristics to the products.
Exposing skincare and body care product ingredients to these sounds through a playback device for an initial recommended duration of 3 minutes, with the understanding that this duration may be adjusted in future applications as scientific understanding evolves. If the sound produced by an instrument is audible to the human ear, it is deemed sufficient for the product components to be within the effective range for infusion.
The claim encompasses a wide range of environmental sounds, including both pure natural sounds and those that have been enhanced, edited, or otherwise modified by humans. Emphasizes the infusion of environmental acoustic elements into skincare products, leveraging the diverse vibrational qualities of natural sounds, whether they are unaltered or human enhanced.

9. Method for Infusing Skin and Body Care Products with Frequencies through Use of Crystals and/or Gemstones, Color Therapy and Spiral Liquid Vortex During the Infusion Process.
This method introduces a unique approach for infusing skin and body care products with enhanced frequency qualities. Initially, water, a key component of the product, undergoes a spiral vortex motion. During this phase, extracts from selected crystals and/or gemstones are introduced. The combination of the vortex motion and gemstone extracts for a minimum of 3 minutes is designed to facilitate a frequency-enhancing process. While whole gemstones are involved only during the infusion phase and are subsequently removed, their extracts remain part of the final product.
Simultaneously, the method incorporates the principles of color therapy by selecting specific colors for the product's packaging and labeling. These colors are not merely for aesthetic purposes but are chosen based on their own frequency properties. The intention is that the colors used in packaging and labeling resonate with and enhance the frequencies introduced during the gemstone extract infusion process.
This synergy between the vibrational qualities of the gemstone extracts, the color therapy elements, and the spiral vortex motion aims to create a product with a distinct frequency profile. It's important to clarify that while this method is focused on infusing the product with specific frequencies through these elements, it does not make claims regarding direct therapeutic or health benefits.

10. Method for Extracting Beneficial Compounds from Botanicals Using Sound Technology and Vortex Motion
A method for extracting beneficial compounds, including but not limited to antioxidants, vitamins, minerals, and phytonutrients, from botanical materials, the method comprising:
Selecting botanical material containing a range of beneficial compounds;
Submerging the selected botanical material in an extraction medium, such as water, oil, esters, alcohol, hydrosols, melted butter, hyaluronic acid, and/or partial or full emulsion; Applying spiral vortex motion to the extraction medium during the extraction process; Simultaneously applying sound waves to the submerged botanical material using sound bowls, gongs, or sound from a playback device; Collecting the extraction medium containing the beneficial compounds post extraction process to incorporate into skin and body care final products.

11. Method for Utilizing Sound-Based Techniques in Skincare and Body Care Product Formulation Process
A method for incorporating various sound-based techniques in the skincare formulation process, comprising: Utilizing sound-producing tools including but not limited to sound bowls, gongs, tuning forks, playback devices, musical instruments, and/or sacred instruments and other similar instruments during the preparation or processing of skincare product ingredients; Engaging these sound-producing tools for purposes such as mixing, infusing, stirring, agitating, or otherwise processing skincare product components; Optionally incorporating sound infusion methods, including singing, vocalizations, or chants, to enhance the formulation process; Wherein the sound-based techniques are employed at any stage of the skincare and/or body care product formulation process, from initial ingredient mixing to final product homogenization.

12. Method for Infusing Skin and Body Care Products with Sound Frequencies Regarding Volume and Infusion Duration
A method for infusing skincare and body care products using sound, characterized by the duration of sound exposure and volume control. The method involves exposing products to sound for durations ranging from milliseconds to several weeks, tailored to the product's specific needs. This flexibility allows for adaptation to various product components and evolving scientific insights.
The volume of sound exposure ranges from barely audible to the highest level that maintains product integrity, covering all intermediate levels.
This claim focuses on the combination of time duration and volume settings, offering a versatile approach for a wide range of skincare and body care products, independent of specific frequency parameters.

13. Method for Infusing Skin and Body Care Products with Sound Frequencies Using a Spiral Vortex
A method for infusing skincare and body care products with sound frequencies, comprising the following steps:
a) Selecting a product component(s) for infusion;
b) Applying sound frequencies to the selected product component(s) using one of the frequency processes outlined above; while simultaneously employing a device to create a spiral vortex within the product component(s);
d) Maintaining the frequency application and spiral vortex movement for the recommended duration of at least 3-15 minutes, with the understanding that this duration may be adjusted in future applications as scientific understanding evolves.
